(19) United States
(12) Patent Application Publication
(10) Pub. No.: US 2004/0249412 A1
(43) Pub. Date: Dec. 9, 2004

(54) Apparatus and methods for puncture site closure

(76) Inventors: Brent W. Snow, Salt Lake City, UT (US); Jeremy W. Snow, North Salt Lake, UT (US)

Correspondence Address: TRASK BRITT, P.O. BOX 2550, SALT LAKE CITY, UT, 84110, US

(21) Appl. No.: 10/454,432

(22) Filed: Jun. 4, 2003

(51) Int. Cl. A61B 17/08

(52) U.S. Cl. 606/213

(57) ABSTRACT

Devices, systems and methods for closing incisions and puncture wounds, such as those trocar openings necessary for port based surgery. An insertion tube is passed through the puncture wound, as by insertion through a trocar, to allow access therethrough. A wound sealing apparatus including a body and a number of attachment elements, such as retaining hooks, is passed from the insertion tube. The attachment elements pierce the fascia around the wound to retain the apparatus in position as the body seals the opening. Actuation may occur through the use of a plunger that extends down a bore of the insertion tube to actuate the apparatus. In some embodiments, actuation rotates a number of arms from the insertion tube to place the attachment elements in the appropriate position.

### FIELD OF THE INVENTION

The present invention relates to the field of surgical instruments and is directed to apparatus and methods for closing and suturing wounds. More particularly it is related, but not necessarily limited, to apparatus and methods for suturing and closing incisions, puncture wounds and the like.

### BACKGROUND

For surgeries using scopes, pointed surgical instruments, called trocars or ports, are used to provide access to body cavities by creating puncture openings allowing access therethrough. For a typical laparoscopic surgery, for example, three or four trocars are used, each creating a corresponding puncture wound in the abdominal wall. Trocars range from about 2 mm to about 18 mm in diameter. For adult patients, the most commonly used are from about 10 to about 12 mm in diameter. For pediatric surgery, trocars of from about 3 mm to about 5 mm in diameter are most commonly used.

Each trocar forms a puncture wound, passing through the skin, subcutaneous fatty tissue, surface fascia, muscle, deep fascia and peritoneum into the underlying cavity, such as the abdominal cavity or the thoracic cavity. Once the surgery is complete, each puncture wound in the abdominal cavity provides an opening through which the peritoneum and intestines may protrude forming a postoperative incisional hernia. In order to reduce the occurrence of postoperative incisional hernia, it is customary to close or suture the trocar openings upon trocar removal. In the thoracic cavity, airtight closures must be formed to alleviate the concern that air may leak into or out of the pleural space, causing the lung to collapse. Similar concerns arise in other body cavities.

Where multiple trocars are used, one or more trocar openings can be sutured using the camera and surgical instrument to observe and return the needle and suture from below. In laparoscopic closure, this allows the practitioner to suture through the abdominal wall while returning the needle using the instrument and monitoring the needle to prevent it from contacting the internal organs. Unfortunately, this method cannot be used for all trocar openings. Once the camera and instrument are removed, at least two trocar openings remain to be closed.

The most common way to close the remaining trocar openings has traditionally been suturing with a curved suture needle. The skin, subcutaneous fat and muscle are retracted from the puncture site. The curved needle and an attached suture are passed through the fascia on one side and back through the fascia on the opposite side. The ends of the suture are then pulled tight and tied to close the opening. Complications may arise from the use of this method. For example, if a patient is obese, several inches of subcutaneous fat must be retracted for the fascia to be isolated. The thick layer of fat causes the fascial tissue to be recessed several inches from the exterior of the abdominal wall, making manipulation of the needle between the thick walls of fat and tissue difficult. Because the procedure involves passing the needle into the body cavity, poor control of the needle may result in damage to the underlying organs, as by puncture of an underlying bowel or inadvertent incorporation of the bowel into the fascial closure of the abdomen.

Attempts have been made to provide instruments for suturing trocar openings that do not require the use of the camera. Typical of these attempts are devices that use an elongated “hook” needle, similar in shape to a shepherd's crook. The hook needle is passed through the puncture wound and then maneuvered to pierce the abdominal wall on the side of the wound. Using the hook needle, suture material is then passed through the wall. This process is repeated on the opposite side of the wound. The suture may be retracted through the puncture wound and drawn together to close the wound. Representative examples of such hooked needle instruments are disclosed in U.S. Pat. No. 5,632,752 to Buelna, U.S. Pat. No. 5,662,663 to Shallman, and U.S. Pat. No. 5,434,69 to Heaven et al., the disclosure of each of which is incorporated by reference herein. Other attempts have involved instruments that use two needles located on either side of the trocar to pierce the abdominal wall from the exterior. Suture is then passed through the needles into the abdominal cavity where it is captured and drawn through the trocar, allowing the wound to be drawn closed. Examples of such devices include U.S. Pat. No. 6,203,554 to Roberts and U.S. Pat. No. 5,476,470 to Fitzgibbons, the disclosure of each of which is incorporated by reference herein. These attempts still require removable needles and/or loops of suture material to be present in the abdomen. These may capture portions of the bowel into the sutures or otherwise injure the internal organs, similar to suturing with a curved needle.

It would be desirable to provide an instrument and methods for closing a puncture wound, such a trocar opening. It would be further desirable for such systems and methods to be insertable and functional through a trocar, resulting in the closure of the wound on trocar removal.

### BRIEF SUMMARY OF THE INVENTION

The present invention is directed to devices, systems and methods for closing incisions and puncture wounds, such as those trocar openings necessary for port based surgery, such as laparoscopy, thorascopy, retroperitoneuoscopy, preperitoneuoscopy, and the like. An insertion tube is passed through the puncture wound, as by insertion through a trocar or port, to allow access therethrough. A wound sealing apparatus including a body and a number of attachment elements, such as retaining hooks, is passed from the insertion tube. The attachment elements pierce the fascia around the wound to retain the apparatus in position as the body seals the opening. Actuation may occur through the use of a plunger that extends down a bore of the insertion tube to actuate the apparatus. In some embodiments, actuation rotates a number of arms from the insertion tube to place the retaining hooks in the appropriate position. A further element may include a sheet member interlocking between the attachment elements to protect further the interior of the wound.

### BRIEF DESCRIPTION OF THE SEVERAL VIEWS OF THE DRAWINGS

The nature of the present invention as well as other embodiments of the present invention may be more clearly understood by reference to the following detailed description, to the appended claims, and to the several drawings herein, wherein:

FIG. 1 is an exploded view of one embodiment of a system for closing a puncture wound;

FIG. 2 is a side view of a portion of the system of FIG. 1, showing details of some components thereof, including one embodiment of a puncture wound sealing apparatus;

FIG. 3 is a side view of the puncture wound sealing apparatus of FIG. 2, in position to close an abdominal puncture wound;

FIG. 4 is a side view of a second embodiment of a puncture wound sealing apparatus, shown in connection with a portion of a system for emplacing the apparatus;

FIG. 5 is a side view of the puncture wound sealing apparatus of FIG. 4, in position to close an abdominal puncture wound;

FIG. 6 is a side view of a third embodiment of a puncture wound sealing apparatus, shown in connection with a portion of a system for emplacing the apparatus; and

FIG. 7 is a side view of the puncture wound sealing apparatus of FIG. 6, in position to close an abdominal puncture wound.

### DETAILED DESCRIPTION

The following describes several embodiments of this invention. It will be appreciated that the examples used herein are illustrative only and do not limit the invention. For example, for the purposes of clarity, the systems and methods of the present invention are discussed in connection with the closure of a puncture wound formed as an abdominal trocar puncture site for a laparoscopic procedure. Such discussion is not intended to limit, and does not limit, the present invention, which may be used to close any suitable wound in any suitable tissue. For example, trocar puncture sites in other tissues created for surgical procedures, such as laparoscopy, thorascopy, retroperitoneuoscopy, preperitoneuoscopy, and the like, may be closed using the methods and systems of the present invention. Similarly, accidental wounds, including, but not limited to, puncture wounds, of appropriate size may be closed using the present invention and all such uses are within the scope of the present invention.

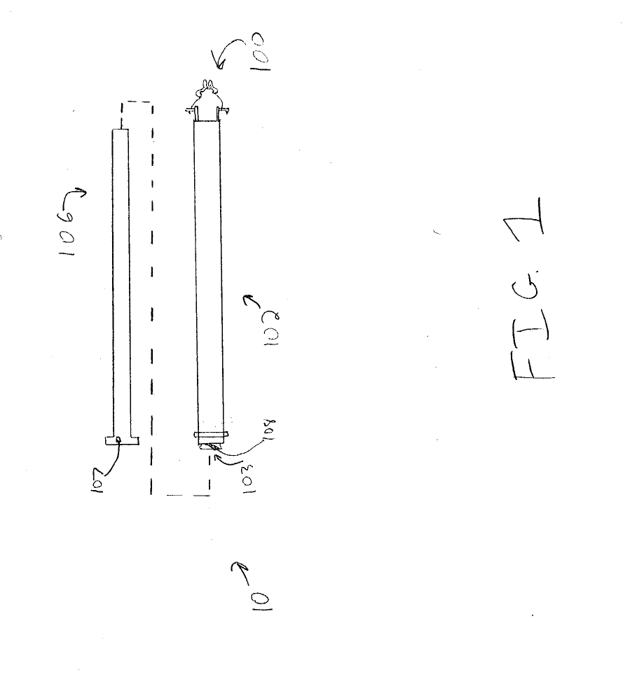

FIG. 1 depicts an exploded view of a system 10 for closing a puncture wound, such a trocar puncture site opening. A wound sealing apparatus 100 is disposed near the distal end of an insertion tube 102. The wound sealing apparatus 100 and distal end of the insertion tube 102 are discussed in further detail in connection with the remaining FIGS. The insertion tube 102 and wound sealing apparatus 100 may be inserted through a hollow trocar 104 (not depicted in FIG. 1) to allow for closure of the puncture site. An appropriately sized, elongated plunger 106 may be inserted through the bore 103 of insertion tube 102 to actuate the wound sealing apparatus 100. A locking mechanism, such as projection 107 interacting with a channel 108 on the insertion tube 102 may be used to retain the plunger 106 in the unactuated position or to guide the plunger 106 during actuation. Insertion tube 102, plunger 106 and the wound sealing apparatus 100 may all be appropriately sized to pass through the bore of a trocar 104 and cover a trocar puncture site, having a wound opening size of from about 2 mm to about 18 mm in diameter. The invention includes different diameter insertion tubes 102, plungers 106, and different sized wound sealing prostheses 100 to accommodate to differently sized wound openings.

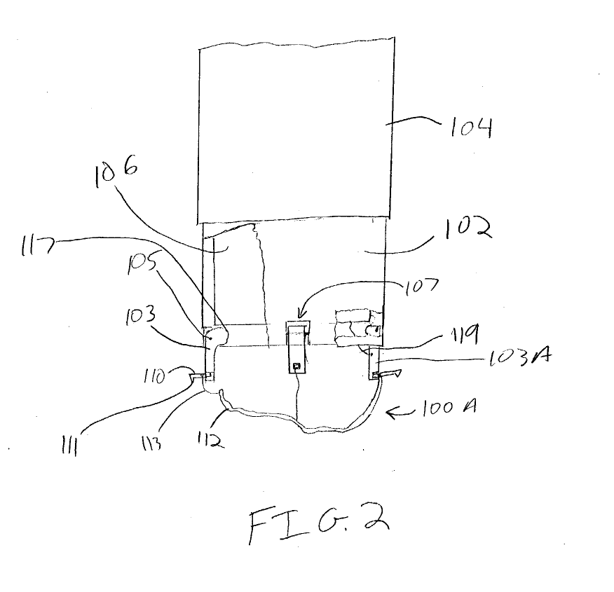

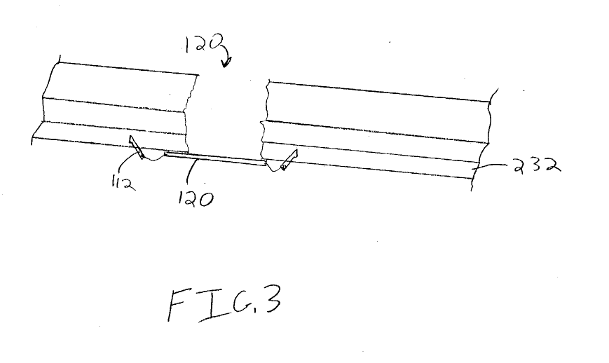

Turning to FIG. 2, details of a first embodiment of a wound sealing apparatus 100A may be seen in connection with the remainder of the wound closure system 10. Insertion tube 102 has been inserted through a trocar 104, such that the distal end thereof protrudes therefrom. The distal end of insertion tube 102 and the wound sealing apparatus 100A thus reside within a patient. In the exemplary abdominal trocar puncture site wound 120 (FIG. 3) usage, the distal end of the insertion tube 102 and wound sealing apparatus 100A reside within the abdominal cavity of the patient. It will be appreciated that although depicted as a circular hollow tube with a sold wall, insertion tube 102 may be any suitable shape, such as circular, ovoid, square, or any other shape in cross-section, and, where appropriate, may have open sides or side openings, consisting of a number of guide members or rails forming the outline of the bore.

Wound sealing apparatus 100A consists of a number of attachment elements, shown as hooks 110, that are used to attach the apparatus 100A to the patient tissues adjacent the puncture wound 120 and a body 112 that is used to close the wound 120. Hooks 110 may be made from any suitable material that is biologically acceptable and may be retained in the body, similar to a surgical staple. For example, hooks 110 may be surgical steel or an acceptable plastic, polymeric or bioabsorbable material. It will be appreciated that any desired number of hooks 110 may be used, surrounding the body 112. For example, embodiments with 3, 4, 6, or any other desired number of hooks 110 may be used. The hooks 110 may be spaced at equal intervals around the body 112. In addition to hooks 110, the attachment elements may comprise pins, staples, needles or any other device capable of attaching the wound sealing apparatus 100 to the fascia or other tissue surrounding a puncture wound.

In the depicted embodiment 100A, the body 112 is formed from a sheet material. Examples of suitable sheet materials include any flexible sheet material that may be used to cover the opening of a puncture wound 120. The sheet material may have elastic properties to allow for better conformation to the puncture site, as tissues relax around the site following trocar 104 removal. Examples include sheets of mesh materials formed from surgically acceptable materials, such as absorbable suture material, or any other commonly used surgical mesh. Commonly used surgical meshes include Gore-Tex™ meshes and biomaterials, polypropylene meshes, such as those offered under the SURGIPRO™ name by United States Surgical of Norwalk, Conn. or under the MARLEX™ name by C. R. Bard, Inc., polyglactin meshes offered by Ethicon, Inc. under the name VICRYL™, polyglycolic acid mesh, such as that available under the name DEXON™, and any other suitable materials. Some mesh materials may be formed from monofilament or multifilament yams and woven, molded or formed using any other known method of forming a prosthetic mesh material. Embodiments using any suitable mesh materials may be used and are within the scope of present invention. Additional sheet materials that may be used include silicone elastomer sheeting and oxidized regenerated cellulose, although it will be appreciated that any suitable sheeting material may be used. For example, a gas-impervious sheet material maybe used in embodiments intended to close puncture wounds to respiratory structures, such as the lungs or chest wall, to allow for an airtight closure to be made.

As shown in FIG. 2, in the undeployed position, each hook 110 is attached to a swivel arm 103 that is rotatably attached to the insertion tube 102. Hook 110 may be attached to the swivel arm 103 by a simple friction fit, residing in a slot located therein. Each swivel arm 103 is attached to the insertion tube 102 in such a manner that it may be actuated to swivel out from an unactuated position therefrom. For example, in the depicted embodiment each swivel arm 103 is disposed on a swivel pin 105 located in a notch 107 formed at the distal end of the insertion tube 102, function as a hinge. In other embodiments, a swivel arm 103 may be formed as a living hinge, or other extension, extending from the insertion tube 102, or may be attached in any other suitable manner. In the unactuated position, the swivel arms 103 may reside within the diameter of the trocar 104. The body 112 of apparatus 100A is in a collapsed position in the undeployed state. In some embodiments, the body 112 may be folded into a desired collapsed conformation. The sheet material of body 112 may be directly attached to each of the hooks 110, or a hook line 113 of suture or other suitable material, may be used to make such an attachment.

To deploy the apparatus 100A, the swivel arms 103 are rotated out from the distal end of the insertion tube 102 bringing hooks 110 into contact with the deep fascia 232 (FIG. 3) of the abdominal wall. Hooks 110 pierce the deep fascia 232 and are retained therein. Barbs 111 or other enlarged retention structures may be used to retain the hooks 110 in the deep fascia 232. The hooks 110 release from the swivel arms 103, as by sliding out of the friction fit channel slots. As swivel arms 103 are actuated, the body 112 unfolds to an expanded position. These actions may occur as the trocar 104 and insertion tube 102 are withdrawn through the puncture wound 120.

As shown in FIG. 2, the swivel arms 103 may be activated by the distal end of plunger 106 pressing upon the actuation structure, such as the enlarged rear portion 117 of the swivel arms 103. This simple blocking and pushing motion may be all that is required. The swivel arms 103 are actuated as the plunger is extended further down the bore of the insertion tube 102. Where present, the channel 108 and projection 107 may interact to guide the plunger to be extended to the correct final depth for actuation.

In other embodiments, a linkage may be established between the plunger 106 and the actuation structure, to allow for additional control, depicted by linkage line 119 that runs from swivel arm 103A to the plunger 106. In embodiments including such linkage, withdrawing the plunger may result in the contraction of the swivel arms. While using such an embodiment, the user may examine the position of the apparatus 100A prior to “setting” the hooks 110 in the fascia 232, then contract and reposition the apparatus if the position is not satisfactory. In other embodiments, the actuation may occur through a rack and pinion mechanism or gears attached to the swivel arms 103 that interact with the plunger 106. Of course, it will be appreciated that embodiments not requiring a plunger 106, where the swivel arms 103 are actuated by other mechanical means, such as an actuator strip sliding down the insertion tube surface, or a motorized swivel arm activated by an electrical current or other signal are within the scope of the present invention.

Once the hooks 110 are set in the deep fascia 232 and the trocar 104 and insertion tube 102 are removed, the sheet material of the body 112 lies over the opening of the puncture wound 120, effectively closing and sealing it from the extrusion of internal structures, as shown in FIG. 3. It will be appreciated that apparatus and methods associated with the embodiment of 100A are not limited to the practice with trocar puncture sites, but may be used to close any suitable wound, including suitable sized accidental punctures. Similarly, although the usage and methods are discussed in connection with abdominal puncture wounds, any suitable wounds in any suitable tissue may be closed using the device.

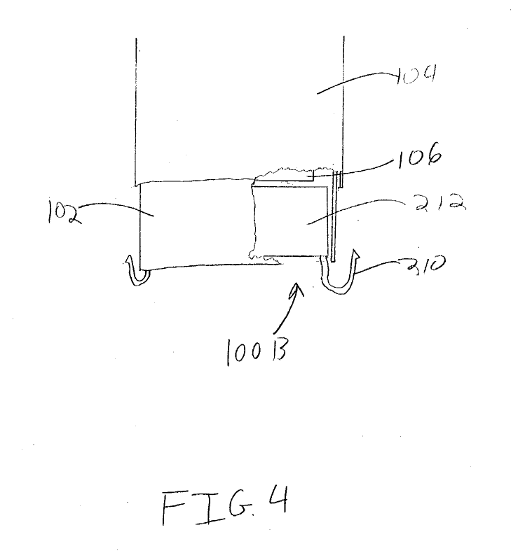

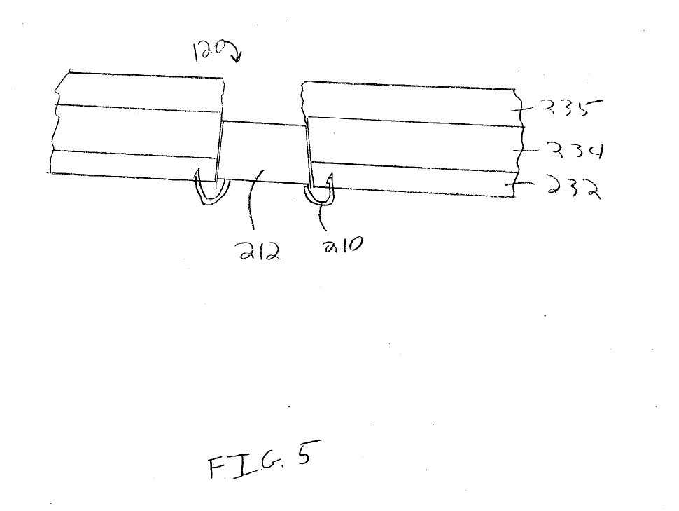

Turning to FIGS. 4 and 5, a second embodiment of a wound sealing apparatus 100B is depicted. Wound sealing apparatus 100B consists of a number of hooks 210 connected to a pledgett body 212. As shown in FIG. 4, the pledgett body 212 may be directly connected to the hooks 210, although embodiments where the hooks 210 are attached to the pledgett body through a short length of line, such as an absorbable suture material or other filament may be used.

At least two attachment elements, shown as hooks 210, located on opposite sides of the pledgett body 212 may be used to position and retain the wound sealing apparatus in the puncture wound 120 site. It will be appreciated that any desired number of hooks may be used, surrounding the pledgett body 212. For example, embodiments with 3, 4, 6, or any other desired number may be used. The hooks 210 may be spaced at equal intervals around the pledgett body 212. As with hooks 110 discussed previously herein, hooks 210 may be formed from any suitable material, including surgical steel.

Where hooks 210 are directly attached to the pledgett body 212, the entire apparatus 100B may be located in the distal end of the insertion tube 102, with the hooks protruding therefrom. In such embodiments, the pledgett body 212 may have a diameter similar to, or only slightly smaller than, that of the bore of the insertion tube 102. In embodiments where the pledgett body 212 is attached to the hooks using an intervening structure, the hooks 210 may be located in setting swivel arms, as discussed previously herein. Where the hooks 210 implanted by swivel arms, the pledgett body 212 may be pushed through the insertion tube 102 prior to hook set and then pulled into the puncture site by a guide cord.

As the trocar 102 and insertion tube 104 are withdrawn, plunger 106 is manipulated to push the pledgett body 212 out of the bore of the insertion tube, once hooks 210 have pierced the internal surface of the abdominal wall. Once trocar 104 and insertion tube 102 are removed, the pledgett body 212 then resides within the abdominal wall retained in position by hooks 210, as shown in FIG. 5. Pledgett body 212 acts a plug sealing the puncture wound 120, which partially contracts to collapse around pledgett body 212. It will be appreciated that a wound sealing apparatus 100B may be used to close any puncture wound and that the closure of an abdominal trocar puncture site is only illustrative. For example, accidental wounds of appropriate size may be closed, as may be trocar puncture sites in other tissues.

As shown in FIG. 5, pledgett body 212 resides within the abdominal wall, with hooks 210 attached to the deep fascia 232 to retain the wound sealing apparatus 100B in position. Pledgett body 212 may pass through any of the various layers of the abdominal wall, including the deep fascia 232, muscle wall 234, and subcutaneous fascia and fat 235. It will be appreciated that the pledgett body 212 need only result in sealing of the deep fascia 232 or the muscle wall 234 to effect closure of the puncture wound 120.

Pledgett body 212 may be formed from a biologically active template material, or substrate. In such embodiments, pledgett body 212 can act as a matrix to allow and encourage the surrounding tissues to grow therethrough and heal the puncture wound 120. Examples of suitable biologically active substances include small intestine submucosa (SIS) and collagen biomatrix, among others. In other embodiments, the pledgett body may be formed from an absorbable suture material.

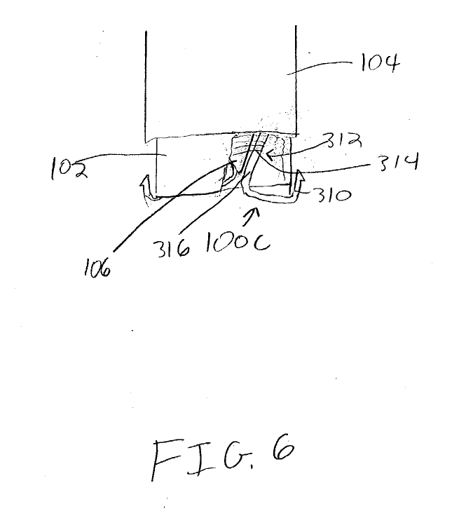

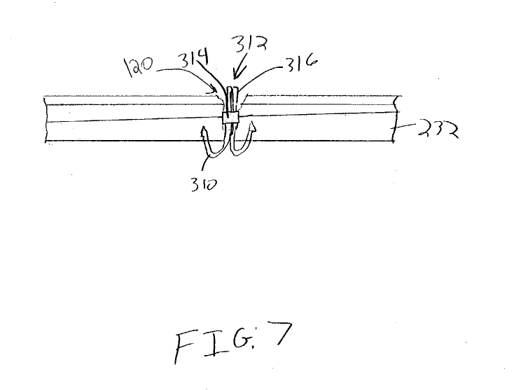

Another embodiment of a wound sealing apparatus 100C is depicted in FIGS. 6 and 7. FIG. 6 shows the apparatus 100C in position in the distal end of the bore of an insertion tube 102 inserted into a trocar 104. Wound sealing apparatus 100C consists of a number of attachment element hooks 310 connected to a body generally indicated at 312. At least two hooks 312, located on opposite sides of the body 312 may be used to position and retain the wound sealing apparatus in the puncture wound 120 site. It will be appreciated that any desired number of hooks may be used. For example, embodiments with 3, 4, 6, or any other desired number may be used. As with hooks 111 and 210 discussed previously herein, hooks 310 may be formed from any suitable material, including surgical steel.

Body 312 is formed from the base shafts 316 of hooks 310 and a contracting mechanism, such as spring 314. The contracting mechanism may be any device or mechanism that is capable of drawing the shafts 316 of the hooks towards one another. For example, a ribbon spring or a coil spring may be wrapped around the shafts 316 that retains the shafts against one another. Another structure, such as a band with contractile or elastic properties may placed around the shafts 316 to draw them together. Strands of a less elastic material wound in conformation to form a contractile band may also be used.

As depicted in FIG. 6, when ready for deployment apparatus 100C maybe located in the distal end of the insertion tube 102, with the hooks 310 protruding therefrom. The distal end of plunger 106 is configured to retain the body 312 in an uncontracted position. This may be accomplished through the use of a slanted, or conical distal end 107 that resides between the shafts 316, holding them apart.

As trocar 104 and insertion tube 102 are withdrawn, plunger 106 may be manipulated to push body 312 out of the bore of the insertion tube 102, as hooks 310 pierce the internal surface of the abdominal wall. The plunger 106 is then withdrawn as trocar 104 and insertion tube 102 are removed, contracting body 312 to close the puncture wound 120. Apparatus 100C is retained in position by hooks 310, as shown in FIG. 7. The contraction of body 312 as the shafts 316 are drawn together contracts the puncture wound 120 around body 312 to achieve closure. Of course, it will be appreciated that wound sealing apparatus 100C may be used to close any puncture wound and that the closure of an abdominal trocar puncture site is only illustrative. For example, accidental puncture wounds of appropriate size may be closed, as may be trocar puncture sites in other tissues.

It will be appreciated that the systems and components of the present invention may be offered as kits for surgical use. One suitable kit would contain an insertion tube 102, plunger 106, and at least one wound sealing apparatus 100, all appropriate sized to function together and accommodate a particular wound size. Each kit may further include an appropriately sized trocar 104 to provide all that is needed for a single laparoscope port in one kit. Each kit may further include an openable package having a top, bottom ,and sides defining a space for containing the surgical components. The package may keep the surgical components sterile until opened for use. Another kit may include a number of wound sealing apparatuses 100, each of which maybe used for closing a single puncture wound. The plunger 106, insertion tube 102 and trocar 104 for use with such a kit may all be reusable with each wound sealing apparatus 100, and may be cleaned and sterilized for use in subsequent operations.

Although the present invention has been shown and described with respect to preferred embodiments, various additions, deletions and modifications that are obvious to a person skilled in the art to which the invention pertains, even if not shown or specifically described herein, are deemed to lie within the scope of the invention as encompassed by the following claims.

What is claimed is:

1. An apparatus for closing a puncture wound, comprising:
a hollow insertion tube sized and adapted to pass through the puncture wound;
a wound sealing apparatus comprising a body and a number of attachment elements attached to the body, the wound sealing apparatus configured to seal a puncture wound upon actuation at a distal end of the insertion tube; and
a plunger configured for actuating the wound sealing apparatus through the hollow portion of the insertion tube.

2. The apparatus of claim 1, where the number of attachment elements attached to the body comprise a number of retaining hooks attached to the body.

3. The apparatus of claim 1, wherein the insertion tube further comprises a plurality of swivel arms rotatably attached to a distal end of the insertion tube for placing the attachment elements in tissue around the puncture wound.

4. The apparatus of claim 3, wherein the body of the wound sealing apparatus comprises a flexible sheet material.

5. The apparatus of claim 4, wherein the flexible sheet material comprises a surgical mesh material.

6. The apparatus of claim 4, wherein the plurality of swivel arms rotatably attached to a distal end of the insertion tube comprises each swivel arm of said plurality being disposed on a retaining pin attaching the swivel arm to the insertion tube.

7. The apparatus of claim 6, wherein the plunger is configured for actuating the wound sealing apparatus by rotating the plurality of swivel arms to attach the attachment elements to tissues around the wound and expand the flexible sheet material of the body of the wound sealing apparatus to an expanded position.

8. The apparatus of claim 1, wherein the body of the wound sealing apparatus comprises a pledgett body for plugging a puncture wound.

9. The apparatus of claim 8, wherein the pledgett body comprises a biologically compatible matrix material.

10. The apparatus of claim 1, wherein the body of the wound sealing apparatus comprises a plurality of shafts, each shaft of the plurality of shafts extending from a hook of a number of retaining hooks comprising the attachment elements and a contracting mechanism.

11. The apparatus of claim 10, wherein the contracting mechanism comprises a spring disposed around the plurality of shafts.

12. A wound sealing apparatus, comprising:
a body for sealing a puncture wound, the body having a number of attached retaining hooks, wherein the body is sized and adapted to put through the puncture wound.

13. The wound sealing apparatus of claim 12, wherein the body of the wound sealing apparatus comprises a flexible sheet material.

14. The wound sealing apparatus of claim 13, wherein the flexible sheet material comprises a surgical mesh material.

15. The wound sealing apparatus of claim 13, wherein each retaining hook of the number of retaining hooks is configured for removable attachment to a swivel arm rotatably attached to a distal end of an insertion tube.

16. The wound sealing apparatus of claim 12, wherein the body of the wound sealing apparatus comprises a pledgett body for plugging a puncture wound.

17. The wound sealing apparatus of claim 16, wherein the pledgett body comprises a biologically compatible matrix material.

18. The wound sealing apparatus of claim 12, wherein the body of the wound sealing apparatus comprises a plurality of shafts, each shaft of the plurality of shafts extending from a hook of number of retaining hooks and a contracting mechanism.

19. The wound sealing apparatus of claim 18, wherein the contracting mechanism comprises a spring disposed around the plurality of shafts.

20. A method of closing a puncture wound, the method comprising:
inserting an elongated hollow insertion tube into a puncture wound;
inserting an actuatable wound sealing apparatus through the insertion tube, wherein a number of retaining hooks of the apparatus upon actuation are deployed into tissues surrounding the puncture wound,
removing the insertion tube from the puncture wound such that the actuated wound sealing apparatus seals the puncture wound.

21. The method according to claim 20, wherein inserting an elongated hollow insertion tube into a puncture wound comprises inserting the insertion tube into a trocar located at a trocar puncture site.

22. The method according to claim 20, wherein inserting an actuatable wound sealing apparatus through the insertion tube, wherein a number of retaining hooks of the apparatus upon actuation are deployed into tissues surrounding the puncture wound comprises rotating a plurality of swivel arms out from the insertion tube to place the retaining hooks in contact with the tissues surrounding the puncture wound.

23. The method according to claim 22, wherein rotating a plurality of swivel arms out from the insertion tube to place the retaining hooks in contact with the tissues surrounding the puncture wound comprises advancing a plunger distally through the hollow portion of the insertion tube to cause the swivel arm to rotate.

24. The method according to claim 20, wherein removing the insertion tube from the puncture wound such that the actuated wound sealing apparatus seals the puncture wound comprises removing the insertion tube to allow a sheet material of the body of the wound sealing apparatus to cover an opening of the puncture wound.

25. The method according to claim 20, wherein removing the insertion tube from the puncture wound such that the actuated wound sealing apparatus seals the puncture wound comprises removing the insertion tube to allow the tissues surrounding the puncture wound to collapse around a pledgett of the body of the wound sealing apparatus.

26. The method according to claim 20, wherein removing the insertion tube from the puncture wound such that the actuated wound sealing apparatus seals the puncture wound comprises removing the insertion tube to allow a contracting mechanism of the wound sealing apparatus to contract, drawing the puncture wound closed.

27. A puncture wound closure kit, comprising:
a hollow insertion tube sized and adapted to pass through a puncture wound;
a wound sealing apparatus comprising a body containing a number of actuatable attachment elements, the wound sealing apparatus sized and configured to seal a puncture wound upon actuation at a distal end of the insertion tube; and
a plunger sized and adapted to fit inside the hollow insertion tube.

28. The puncture wound closure kit of claim 27, wherein the number of actuatable attachment elements comprises a number of retaining hooks.

29. The puncture wound closure kit of claim 27, wherein the insertion tube further comprises a plurality of swivel arms rotatably attached to a distal end of the insertion tube for placing the attachment elements in tissue around the puncture wound.

30. The puncture wound closure kit of claim 29, wherein the body of the wound sealing apparatus comprises a flexible sheet material.

31. The puncture wound closure kit of claim 30, wherein the flexible sheet material comprises a surgical mesh material.

32. The puncture wound closure kit of claim 30, wherein the plurality of swivel arms rotatably attached to a distal end of the insertion tube comprises each swivel arm of said plurality being disposed on a retaining pin attaching the swivel arm to the insertion tube.

33. The puncture wound closure kit of claim 32, wherein the plunger is configured for actuating the wound sealing apparatus by rotating the plurality of swivel arms to attach the attachment elements to tissues around the wound and expand the flexible sheet material of the body of the wound sealing apparatus to an expanded position.

34. The puncture wound closure kit of claim 27, wherein the body of the wound sealing apparatus comprises a pledgett body for plugging a puncture wound.

35. The puncture wound closure kit of claim 34, wherein the pledgett body comprises a biologically compatible matrix material.

36. The puncture wound closure kit of claim 28, wherein the body of the wound sealing apparatus comprises a plurality of shafts, each shaft of the plurality of shafts extending from a hook of the number of retaining hooks and a contracting mechanism.

37. The puncture wound closure kit of claim 36, wherein the contracting mechanism comprises a spring disposed around the plurality of shafts.

38. The puncture wound closure kit of claim 27, further comprising a trocar appropriately sized for pass the hollow insertion tube therethrough.
